(19) United States
(12) Patent Application Publication
(10) Pub. No.: US 2023/0183703 A1
(43) Pub. Date: Jun. 15, 2023

(54) COMPOSITIONS AND METHODS FOR TREATING AND DETECTING CORONAVIRUS INFECTION

(71) Applicants: Hemayet ULLAH, Washington, DC (US); Saadyah Eliezer AVERICK, Pittsburgh, PA (US); HOWARD UNIVERSITY, Washington, DC (US)

(72) Inventor: Hemayet ULLAH, Washington, DC (US)

(73) Assignee: HOWARD UNIVERSITY, Washington, DC (US)

(21) Appl. No.: 17/925,474

(22) PCT Filed: May 14, 2021
(86) PCT No.: PCT/US2021/032425

§ 371 Date: Nov. 15, 2022

Related U.S. Application Data
(60) Provisional application No. 63/025,613, filed on May 15, 2020.

(51) Int. Cl. C12N 15/113; G01N 33/569

(52) U.S. Cl. CPC C12N 15/1131; G01N 33/56983; G01N 2333/165; C12N 2310/11

(57) ABSTRACT

The present disclosure provides compositions and methods for treating and detecting coronavirus infection, and in particular embodiments, provides compositions and methods for treating and detecting SARS-CoV-2 infection. The present disclosure also relates to the production of compositions for treating and detecting coronavirus infection, and in particular embodiments, to the production of compositions for treating and detecting SARS-CoV-2 infection.

### STATEMENT REGARDING FEDERALLY SPONSORED RESEARCH OR DEVELOPMENT

Not applicable.

### SEQUENCE LISTING

The present application contains a Sequence Listing that is hereby incorporated by reference in its entirety.

### TECHNICAL FIELD

The present disclosure relates generally to compositions and methods for treating and detecting virus infection, and in particular embodiments, relates to compositions and methods for treating and detecting coronavirus infection, including SARS-CoV-2 infection. The disclosure also relates to the production of compositions for treating and detecting virus infection, and in particular embodiments, to the production of compositions for treating and detecting coronavirus infection, including SARS-CoV-2 infection.

### BACKGROUND

As of 2021, SARS-CoV-2 has infected more than one-hundred and fifty million individuals worldwide, resulting in more than three million deaths to date. Although the development of vaccines against SARS-CoV-2 has been rapid, there remain no effective treatments against the virus. Hence, for individuals who have contracted SARS-CoV-2; or who are unable to be vaccinated because of underlying health conditions or personal reasons, for example, there remains an urgent need for an effective treatment that is able to inhibit proliferation of the virus. Additionally, because of the intrinsic ability of SARS-CoV-2 to accumulate genomic mutations at a high rate, it is conceivable that future SARS-CoV-2 variants may be resistant to existing vaccines, or vaccines in the late stages of development. Accordingly, in addition to vaccination, there remains an urgent need for effective and durable treatments against SARS-CoV-2.

At present, individuals infected with SARS-CoV-2 generally receive supportive care, such as supplemental oxygen, mechanical ventilation, and steroids. None of these supportive therapies target or inhibit SARS-CoV-2, and their success ultimately relies upon the host's immune system to control the virus. A therapy specifically targeted at SARS-CoV-2 is therefore desirable.

However, the identification of a suitable drug target for SARS-CoV-2, much less the identification of a drug that specifically binds that target, is not straightforward; because existing antiviral drugs (such as nucleoside analogs, for example) often exhibit significant side effects against the host, their use can be problematic. Additionally, viral mismatch repair mechanisms may limit the analogs' effectiveness. Accordingly, the development of a treatment that specifically targets SARS-CoV-2, but which shows little or no serious side effects against the host, is especially desirable.

Additionally, by targeting a target unique to SARS-CoV-2, the same targeting mechanism can also be used to detect the presence of SARS-CoV-2 virus in a sample. Therefore, nucleic acids and compositions of the present disclosure also have utility in the field of SARS-CoV-2 detection, diagnosis, and management.

### SUMMARY OF THE INVENTION

The present disclosure provides novel viral targets that, when inhibited, can potently inhibit viral proliferation. In particular, the viral targets are present in coronaviruses, including SARS-CoV-2. In embodiments herein, the novel viral targets are present in a region that is not amenable to mutation (e.g., because mutations in the region would disrupt the viral replication cycle). In certain embodiments, the viral target is a 5′-poly-uracil (“poly(U)”) sequence present in a negative-sense RNA molecule transcribed from a coronavirus positive-sense viral genome, such as the SARS-CoV-2 positive-sense viral genome. Because the 5′-polyU is indispensable for the virus, any drugs developed targeting the 5′-polyU will be durable (virus will not be able to evade the drugs by mutating)

The present disclosure further provides nucleic acid molecules, and compositions containing such nucleic acid molecules, that hybridize to the 5′-poly(U) sequence in a negative-sense RNA molecule transcribed from a coronavirus positive-sense viral genome, such as the SARS-CoV-2 positive-sense viral genome.

Non-limiting embodiments of the disclosure include as follows.

[1] An agent that binds to a negative-strand ribonucleic acid (RNA) molecule produced by a coronavirus, preferably SARS-CoV-2, wherein said agent is capable of binding to a 5′-poly(U) stretch in said negative-strand RNA molecule, and wherein said agent is selected from the group consisting of a nucleic acid, an antibody, a protein, an aptamer, and a low molecular weight compound.

[2] A nucleic acid molecule that binds to a negative-strand ribonucleic acid (RNA) molecule produced by a coronavirus, preferably SARS-CoV-2, wherein said nucleic acid molecule comprises a poly(A) stretch that is between 5-50 bases in length, and wherein said poly(A) stretch is capable of hybridizing to a 5′-poly(U) stretch in said negative-strand RNA molecule.

[3] The nucleic acid molecule of [2], wherein the nucleic acid molecule further comprises a 5′-end linker region that comprises a sequence complementary to a sequence at the 5′-end of the negative-strand RNA that starts immediately after the poly(U) stretch.

[4] The nucleic acid molecule of [3], wherein the linker sequence comprises GGAGAATGAC (nucleotides 1-10 of SEQ ID NO: 1).

[5] The nucleic acid molecule of [4], wherein said nucleic acid molecule comprises a nucleotide sequence selected from the group consisting of SEQ ID NO: 1 and SEQ ID NO: 2.

[6] A composition comprising the nucleic acid molecule of any one of [2]-[5], wherein said composition further comprises a delivery vehicle.

[7] The composition of [6], wherein the delivery vehicle is a polymer.

[8] The composition of [7], wherein the polymer is selected from the group consisting of a polyethylenimine polymer and a linear poly methacrylate cationic polymer.

[9] The composition of [8], wherein the polyethylenimine polymer is a branched polyethylenimine polymer.

[10] The composition of [6], wherein the delivery vehicle is selected from the group consisting of a lipid, a liposome, a hydrogels, a cyclodextrin, poly(lactic-co-glycolic)acid (PLGA), a microsphere, a nanocapsule, and a protein.

[11] The nucleic acid molecule of [2] or [3], wherein the nucleic acid molecule contains at least one of a modified base, a base analog, and an abasic site.

[12] The nucleic acid molecule of any of one [2]-[5], wherein the nucleic acid molecule is conjugated to a heterologous molecule.

[13] The nucleic acid molecule of [12], wherein the heterologous molecule is a detectable label.

[14] The nucleic acid molecule of [12], wherein the heterologous molecule is a molecule that targets the nucleic acid molecule to a cell or intracellular compartment thereof.

[15] A method for treating a coronavirus infection, preferably SARS-CoV-2 infection, comprising administering to a subject in need thereof the nucleic acid molecule of any one of [2]-[5] and [11]-[14].

[16] A method for treating a coronavirus infection, preferably SARS-CoV-2 infection, comprising administering to a subject in need thereof the composition of any one of [6]-[10].

[17] A method for treating a coronavirus infection, preferably SARS-CoV-2 infection, comprising administering to a subject in need thereof, an agent that binds to a 5′-poly(U) sequence in a negative-strand ribonucleic acid (RNA) molecule produced by a coronavirus, preferably SARS-CoV-2.

[18] The method of [17], wherein said agent is selected from the group consisting of a nucleic acid, an antibody, an aptamer, a protein, and a low molecular weight compound.

[19] A method of blocking a 5′-poly(U) sequence in a negative-strand ribonucleic acid (RNA) molecule produced by a coronavirus, preferably SARS-CoV-2, wherein said method comprises contacting the agent of [1] with a negative-strand ribonucleic acid (RNA) molecule produced by a coronavirus, preferably SARS-CoV-2.

[20] A method of blocking a 5′-poly(U) sequence in a negative-strand ribonucleic acid (RNA) molecule produced by a coronavirus, preferably SARS-CoV-2, wherein said method comprises contacting the nucleic acid molecule of any one of [2]-[5] and [11]-[14] with a negative-strand ribonucleic acid (RNA) molecule produced by a coronavirus, preferably SARS-CoV-2.

[21] A method of blocking a 5′-poly(U) sequence in a negative-strand ribonucleic acid (RNA) molecule produced by a coronavirus, preferably SARS-CoV-2, wherein said method comprises contacting the composition of any one of [6]-[10] with a negative-strand ribonucleic acid (RNA) molecule produced by a coronavirus, preferably SARS-CoV-2.

[22] The nucleic acid of [2], wherein the poly(A) stretch is between 20-30 bases in length.

[23] A method of detecting a coronavirus, preferably SARS-CoV-2, comprising detecting the presence of a negative-strand ribonucleic acid (RNA) molecule produced by a coronavirus, preferably SARS-CoV-2, in a sample, wherein said negative-strand RNA comprises a 5′-poly(U) stretch.

[24] The method of [23], wherein said negative-strand RNA is detected using a nucleic acid amplification reaction, and wherein said nucleic acid amplification reaction amplifies a region that contains all or a part of said poly(U) stretch.

[25] The method of [24], wherein said nucleic acid amplification reaction comprises use of an oligo(dT) primer.

[26] The method of [25], wherein said nucleic acid amplification reaction further comprises use of an oligonucleotide primer comprising the sequence of SEQ ID NO: 3.

[27] The method of [24], wherein said nucleic acid amplification reaction amplifies all or a portion of the region corresponding to SEQ ID NO: 4.

[28] The nucleic acid of [2], wherein the nucleic acid comprises DNA, RNA, or DNA and RNA.

[29] The nucleic acid of [28], wherein the nucleic acid comprises at least one of a modified base, a base analog, or an abasic site.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE FIGURES

The features of the invention are set forth with particularity in the appended claims. A better understanding of the features and advantages of the present invention will be obtained by reference to the following detailed description that sets forth illustrative embodiments, in which the principles of the invention are utilized, and the accompanying figures. The figures are not proportionally rendered, nor are they to scale. The locations of indicators are approximate.

FIG. 1 illustrates the organization of the SARS-CoV-2 genome.

FIG. 2 illustrates the organization of SARS-CoV-2 negative-strand intermediates.

FIG. 3 illustrates the structure of branched polyethylenimine (bPEI).

### DETAILED DESCRIPTION OF THE INVENTION

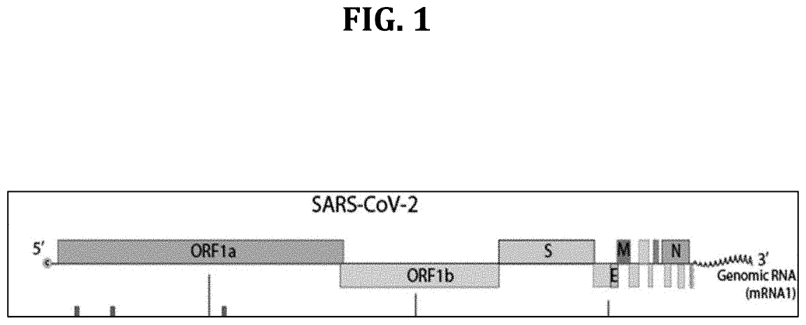

SARS-CoV-2, the causative agent of COVID-19, is an enveloped RNA virus within the family Coronaviridae, and contains a single-stranded, positive-sense (+), RNA genome. The organization of the SARS-CoV-2 genome is depicted in FIG. 1. This is in contrast to the human genome, which is double-stranded DNA. Infection of a cell with the SARS-CoV-2 virus begins with attachment of the SARS-CoV-2 spike (S) protein to its cognate cellular receptor (angiotensin-converting enzyme 2; ACE2), followed by internalization, fusion with the endosomal membrane and uncoating, and translation of viral non-structural proteins (encoded by the single-stranded, positive-sense (+), RNA genome of SARS-CoV-2) to form the viral replicase complex. Following assembly of viral replicase complexes, viral RNA synthesis ensues, which results in the production of both genomic and sub-genomic RNAs (the sub-genomic RNAs are used as mRNAs for the translation of structural and accessory genes). The sub-genomic mRNA cannot serve as templates for negative-strand synthesis, because they lack the 5′—replication signal.

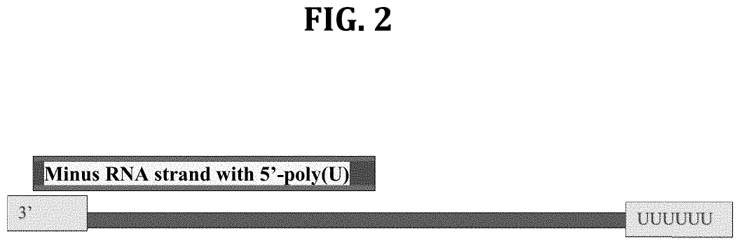

The genomic and sub-genomic RNAs are produced using negative-strand intermediates that contain, amongst others, 5′-polyuridine (poly(U)) sequences complementary to the polyadenylated tail of the single-stranded, positive-sense (+), RNA genome of SARS-CoV-2. Similar to eukaryotic mRNA, the SARS-CoV-2 genome is both 5′-capped, and 3′-polyadenylated (in a template-dependent fashion). The organization of the SARS-CoV-2 negative-strand intermediates is depicted in FIG. 2.

The negative-strand intermediates are synthesized throughout infection (via RNA-dependent RNA polymerase), although their synthesis declines after about six-or seven hours post-infection. In contrast, human cells do not typically produce any negative-strand RNA molecules, and therefore, 5′-poly(U)-containing nucleic acids are typically absent from normal human cells (although some small RNA transcripts synthesized by host RNA polymerase III contain a short stretch of poly(U) at only the 3′-end).

As described herein, the present inventors have demonstrated that targeting the 5′-poly(U) tract of the negative-strand intermediates inhibits SARS-CoV-2 viral replication (blocking the poly(U) tract prevents genomic and sub-genomic mRNA synthesis). Moreover, because 5′-poly(U)-containing nucleic acids are typically absent from normal human cells, blocking the 5′-poly(U) tract of the viral negative-strand intermediates is not associated with off-target effects.

To further enhance targeting and specificity, and to further reduce off-target effects, the 5′-poly(U) sequences may be targeted using a polynucleotide that comprises a poly(A) sequence and which further comprises a 5′-end linker region that comprises a sequence complementary to a sequence at the 5′-end of the negative-strand viral RNA that starts immediately after the poly(U) stretch. In this configuration, both the poly(A) sequence and the 5′-end linker region sequence will hybridize to the SARS-CoV-2 negative-strand intermediates.

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Abbas et al. (Cellular and Molecular Immunology, 2017, 9th Edition, Elsevier, ISBN 978-0323479783); Butterfield et al. (Cancer Immunotherapy Principles and Practice, 2017, 1st Edition, Demos Medical, ISBN 978-1620700976); Kenneth Murphy (Janeway's Immunobiology, 2016, 9th Edition, Garland Science, ISBN 978-0815345053); Stevens et al. (Clinical Immunology and Serology: A Laboratory Perspective, 2016, 4th Edition, Davis Company, ISBN 978-0803644663); E. A. Greenfield (Antibodies: A Laboratory Manual, 2014, Second edition, Cold Spring Harbor Laboratory Press, ISBN 978 936113-81-1); R. I. Freshney (Culture of Animal Cells: A Manual of Basic Technique and Specialized Applications, 2016, 7th Edition, Wiley-Blackwell, ISBN 978-1118873656); C. A. Pinkert (Transgenic Animal Technology, Third Edition: A Laboratory Handbook, 2014, Elsevier, ISBN 978-0124104907); H. Hedrich (The Laboratory Mouse, 2012, Second Edition, Academic Press, ISBN 978-0123820082); Behringer et al. (Manipulating the Mouse Embryo: A Laboratory Manual, 2013, Fourth Edition, Cold Spring Harbor Laboratory Press, ISBN 978-1936113019); McPherson et al. (PCR 2: A Practical Approach, 1995, IRL Press, ISBN 978-0199634248); J. M. Walker (Methods in Molecular Biology (Series), Humana Press, ISSN 1064-3745); Rio et al. (RNA: A Laboratory Manual, 2010, Cold Spring Harbor Laboratory Press, ISBN 978-0879698911); Methods in Enzymology (Series), Academic Press; Green et al. (Molecular Cloning: A Laboratory Manual, 2012, Fourth Edition, Cold Spring Harbor Laboratory Press, ISBN 978-1605500560); and G. T. Hermanson (Bioconjugate Techniques, 2013, Third Edition, Academic Press, ISBN 978-0123822390).

A “linker region sequence,” “linker sequence,” and “linker polynucleotide” are used interchangeably herein and refer to a sequence of one or more nucleotides covalently attached to a first nucleic acid sequence (e.g., 5′-linker nucleotide sequence-first nucleic acid sequence-3′). In some embodiments, a linker nucleotide sequence connects two separate nucleic acid sequences to form a single polynucleotide (e.g., 5′-first nucleic acid sequence-linker nucleotide sequence-second nucleic acid sequence-3′). Other examples of linker sequences include, but are not limited to, 5′-first nucleic acid sequence-linker nucleotide sequence-3′, and 5′-linker nucleotide sequence-first first nucleic acid sequence-linker nucleotide sequence-3′. In some embodiments, the linker nucleotide sequence can be a single-stranded nucleotide sequence of unpaired nucleic acid bases that do not interact with each other through hydrogen bond formation to create a secondary structure. In some embodiments, a linker element nucleotide sequence can be about 100 or less, about 90 or less, about 80 or less, about 70 or less, about 60 or less, about 50 or less, about 40 or less, about 30 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, about 3 or less, or about 2 or less, bases in length.

The terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of the same molecule is present.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

As used herein, “hydrogen bonding,” “hydrogen-base pairing,” and “hydrogen bonded” are used interchangeably and refer to canonical hydrogen bonding and non-canonical hydrogen bonding including, but not limited to, “Watson-Crick-hydrogen-bonded base pairs” (W—C-hydrogen-bonded base pairs or W—C hydrogen bonding); “Hoogsteen-hydrogen-bonded base pairs” (Hoogsteen hydrogen bonding); and “wobble-hydrogen-bonded base pairs” (wobble hydrogen bonding). W—C hydrogen bonding, including reverse W—C hydrogen bonding, refers to purine-pyrimidine base pairing, that is, adenine:thymine, guanine:cytosine, and uracil: adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W—C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine:uracil, inosine (hypoxanthine):uracil, inosine:adenine, and inosine:cytosine. Wobble base interaction are also known to occur between inosine:thymine and inosine:guanine. Inosine bases and deoxy inosine bases can be referred to as “universal pairing bases” as they are capable of hydrogen bonding with the canonical DNA and RNA bases (see, e.g., FIG. 7). See also Watkins et al. (Nucleic Acid Research, 2005, 33(19):6258-67). Rules for canonical hydrogen bonding and non-canonical hydrogen bonding are known to those of ordinary skill in the art. See, e.g., R. F. Gesteland (The RNA World, Third Edition (Cold Spring Harbor Monograph Series), 2005, Cold Spring Harbor Laboratory Press, ISBN 978-0879697396); R. F. Gesteland (The RNA World, Second Edition (Cold Spring Harbor Monograph Series), 1999, Cold Spring Harbor Laboratory Press, ISBN 978-0879695613); R. F. Gesteland (The RNA World, First Edition (Cold Spring Harbor Monograph Series), 1993, Cold Spring Harbor Laboratory Press, 978-0879694562) (see, e.g., Appendix 1: Structures of Base Pairs Involving at Least Two Hydrogen Bonds, I. Tinoco); W. Saenger (Principles of Nucleic Acid Structure, 1988, Springer International Publishing AG, ISBN 978-O-387-90761-1); S. Neidle (Principles of Nucleic Acid Structure, 2007, First Edition, Academic Press, ISBN 978-01236950791).

“Connect,” “connected,” and “connecting” are used interchangeably herein, and refer to a covalent bond or a non-covalent bond between two macromolecules (e.g., polynucleotides, proteins, and the like).

As used herein, the terms “nucleic acid sequence,” “nucleotide sequence,” and “oligonucleotide” are interchangeable and refer to a polymeric form of nucleotides. As used herein, the term “polynucleotide” refers to a polymeric form of nucleotides that has one 5′ end and one 3′ end and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Woburn, Mass.) nucleosides, glycol nucleic acid, bridged nucleic acids, and morpholino structures.

As used herein, the term “poly(U)” refers to a plurality of continuous uracil bases. Similarly, the term “poly(A)” refers to a plurality of continuous adenine bases. It is understood that a continuous stretch of poly(U) or poly(A), for example, may contain some modified bases, base analogs, or abasic sites, and still be considered a poly(U) or poly(A), respectively.

As used herein, the terms “abasic,” “abasic site,” “abasic nucleotide,” and “apurinic/apyrimidinic site,” are used interchangeably and refer to a site in a nucleotide sequence that lacks the purine or a pyrimidine base. In certain embodiments, abasic sites comprise a deoxyribose site. In other embodiments, abasic sites comprise a ribose site. In yet further embodiments, abasic sites comprise a modified backbone, such as a pentose ring with a 1′ hydroxyl group. An abasic site cannot form hydrogen base pair bonding with a complementary nitrogen base of a DNA or RNA nucleotide, because it does not contain a nitrogen base.

As used herein, the term “base analog” refers to a compound having structural similarity to a canonical purine or pyrimidine base occurring in DNA or RNA. The base analog may contain a modified sugar and/or a modified nucleobase, as compared to a purine or pyrimidine base occurring naturally in DNA or RNA. In some embodiments, the base analog is inosine or deoxyinosine, such as 2′-deoxyinosine. In other embodiments, the base analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the base analog may be selected from the group consisting of 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. The term “base analog” also includes, for example, 2′-deoxyribonucleosides, 2′-ribonucleosides, 2′-deoxyribonucleotides or 2′-ribonucleotides, wherein the nucleobase is a substituted hypoxanthine. For instance, the substituted hypoxanthine may be substituted with a halogen, such as fluorine or chlorine. In some embodiments, the base analog may be a fluoroinosine or a chloroinosine, such as 2-chloroinosine, 6-chloroinosine, 8-chloroinosine, 2-fluoroinosine, 6-fluoroinosine, or 8-fluoroinosine. In other embodiments, the base analog is deoxyuradine. In other embodiments the base analog is a nucleic acid mimic (such as, for example, artificial nucleic acids and xeno nucleic acids (XNA).

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at the 5′-end or 3′-end sequences of a polynucleotide sequence to inhibit exonuclease degradation. Placement of phosphorothioate bonds throughout an entire oligonucleotide helps reduce degradation by endonucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., www.ucalgary.ca/dnalab/synthesis/-modifications/linkages). A 3′-3′ linkage at a terminus of a polynucleotide stabilizes the polynucleotide to exonuclease degradation by creating an oligonucleotide having two 5′-OH termini but lacking a 3′-OH terminus. Typically, such polynucleotides have phosphoramidite groups on the 5′-OH position and a dimethoxytrityl (DMT) protecting group on the 3′-OH position. Normally, the DMT protecting group is on the 5′-OH and the phosphoramidite is on the 3′-OH.

Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is typically between about 90% identity and 100% identity over the length of the reference polypeptide, for example, about 90% identity or higher, preferably about 95% identity or higher, more preferably about 98% identity or higher. A moderate degree of sequence identity between two polynucleotides or two polypeptides is typically between about 80% identity to about 85% identity, for example, about 80% identity or higher, preferably about 85% identity over the length of the reference polypeptide. A low degree of sequence identity between two polynucleotides or two polypeptides is typically between about 50% identity and 75% identity, for example, about 50% identity, preferably about 60% identity, more preferably about 75% identity over the length of the reference polypeptide.

For instance, a nucleic acid sequence of the present disclosure may have a particular sequence identity to a sequence complementary to a poly(U) region, and/or a particular sequence identity to a sequence complementary to a sequence at the 5′-end of the negative-strand RNA that starts immediately after the poly(U) stretch. This sequence identity may be, for example, 25% or more, 50% or more, 75% or more, 80% or more, 85% or more, 90% or more, 91% or more, 92% or more, 93% or more, 94% or more, 95% or more, 96% or more, 97% or more, 98% or more, or 99% or more.

As used herein, “hybridization,” “hybridize,” or “hybridizing” is the process of combining two complementary single-stranded DNA or RNA molecules so as to form a single double-stranded molecule (DNA/DNA, DNA/RNA, RNA/RNA) through hydrogen base pairing. Hybridization stringency is typically determined by the hybridization temperature and the salt concentration of the hybridization buffer; e.g., high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tₘ; moderate stringency, approximately 0.16M to approximately 0.33M salt, hybridization temperature 20° C. to 29° C. below Tₘ; and low stringency, approximately 0.33M to approximately 0.82M salt, hybridization temperature 40° C. to 48° C. below Tₘ. Tₘ of duplex nucleic acid sequences is calculated by standard methods well-known in the art. See, e.g., Maniatis et al. (Molecular Cloning: A Laboratory Manual, 1982, Cold Spring Harbor Laboratory Press: New York); Casey et al. (Nucleic Acids Research, 1977, 4:1539-1552); Bodkin et al. (Journal of Virological Methods, 1985, 10(1):45-52); and Wallace et al. (Nucleic Acids Research, 1981, 9(4):879-894). Algorithm prediction tools to estimate Tₘ are also widely available. High stringency conditions for hybridization typically refer to conditions under which a polynucleotide complementary to a target sequence predominantly hybridizes with the target sequence, and substantially does not hybridize to non-target sequences. Typically, hybridization conditions are of moderate stringency, preferably high stringency.

As used herein, “complementarity” refers to the ability of a nucleic acid sequence to form hydrogen bonds with another nucleic acid sequence (e.g., through canonical Watson-Crick base pairing). A percent complementarity indicates the percentage of residues in a nucleic acid sequence that can form hydrogen bonds with a second nucleic acid sequence. If two nucleic acid sequences have 100% complementarity, the two sequences are perfectly complementary, i.e., all of the contiguous residues of a first polynucleotide hydrogen bond with the same number of contiguous residues in a second polynucleotide.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

Regulatory elements include those that direct constitutive, inducible, and repressible expression of a nucleotide sequence in many types of host cells and those that direct expression of the nucleotide sequence only in certain host cells (e.g., tissue-specific regulatory sequences). In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart et al. (Cell, 1985, 41:521-530)), the SV40 promoter, the dihydrofolate reductase promoter, the (3-actin promoter, the phosphoglycerol kinase (PGK) promoter, and the EF1a promoter. It will be appreciated by those skilled in the art that the design of an expression vector may depend on such factors as the choice of the host cell to be transformed, the level of expression desired, and the like. A vector can be introduced into host cells to thereby produce transcripts, proteins, or peptides, including fusion proteins or peptides, encoded by nucleic acid sequences as described herein.

“Gene” as used herein refers to a polynucleotide sequence comprising exons and related regulatory sequences. A gene may further comprise introns and/or untranslated regions (UTRs).

As used herein, the term “operably linked” refers to polynucleotide sequences or amino acid sequences placed into a functional relationship with one another. For example, regulatory sequences (e.g., a promoter or enhancer) are “operably linked” to a polynucleotide encoding a gene product if the regulatory sequences regulate or contribute to the modulation of the transcription of the polynucleotide. Operably linked regulatory elements are typically contiguous with the coding sequence. However, enhancers can function if separated from a promoter by up to several kilobases or more. Accordingly, some regulatory elements may be operably linked to a polynucleotide sequence but not contiguous with the polynucleotide sequence. Similarly, translational regulatory elements contribute to the modulation of protein expression from a polynucleotide.

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, the term “modulate” refers to a change in the quantity, degree or amount of a function. Thus, “modulation” of gene expression includes both gene activation and gene repression. Modulation can be assayed by determining any characteristic directly or indirectly affected by the expression of the target gene. Such characteristics include, for example, changes in RNA or protein levels, protein activity, product levels, expression of the gene, or activity level of reporter genes.

“Vector” and “plasmid” as used herein refer to a polynucleotide vehicle to introduce genetic material into a cell. Vectors can be linear or circular. Vectors can contain a replication sequence capable of effecting replication of the vector in a suitable host cell (e.g., an origin of replication). Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art. The four major types of vectors are plasmids, viral vectors, cosmids, and artificial chromosomes. Typically, vectors comprise an origin of replication, a multicloning site, and/or a selectable marker. An expression vector typically comprises an expression cassette. By “recombinant virus” is meant a virus that has been genetically altered, e.g., by the addition or insertion of a heterologous nucleic acid construct into a viral genome or portion thereof.

As used herein, “expression cassette” refers to a polynucleotide construct generated using recombinant methods or by synthetic means and comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, the term “between” is inclusive of end values in a given range (e.g., between about 1 and about 50 nucleotides in length includes 1 nucleotide and 50 nucleotides.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology. Furthermore, essentially any polypeptide or polynucleotide is available from commercial sources.

The terms “fusion protein” and “chimeric protein” as used herein refer to a single protein created by joining two or more proteins, protein domains, or protein fragments that do not naturally occur together in a single protein.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description. In some embodiments, a 5′ terminus, a 3′ terminus, or a 5′ terminus and a 3′ terminus can comprise one or more moieties.

The terms “modified protein,” “mutated protein,” “protein variant,” and “engineering protein” as used herein typically refers to a protein that has been modified such that it comprises a non-native sequence (i.e., the modified protein has a unique sequence compared to an unmodified protein).

The terms “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent, refer to a sufficient amount of the composition or agent to provide the desired response. Preferably, the effective amount will prevent, avoid, or eliminate one or more harmful side-effects. The exact treatment amount required will vary from subject to subject, depending on the species, age, and general condition of the subject, the severity of the condition being treated, and the particular treatment used, mode of administration, and the like. An appropriate “effective” amount in any individual case may be determined by one of ordinary skill in the art using routine experimentation.

“Treatment” or “treating” a particular disease, such as COVID-19, includes: (1) preventing the disease, for example, preventing the development of the disease or causing the disease to occur with less intensity in a subject that may be predisposed to the disease, but does not yet experience or display symptoms of the disease; (2) inhibiting the disease, for example, reducing the rate of development, arresting the development or reversing the disease state; and/or (3) relieving symptoms of the disease, for example, decreasing the number of symptoms experienced by the subject.

“Transformation” as used herein refers to the insertion of an exogenous polynucleotide into a host cell, irrespective of the method used for insertion. For example, transformation can be by direct uptake, transfection, infection, and the like. Nucleic acids and polynucleotides can be introduced into the cell using, for example, viral vectors, nucleofection, gene gun, sonoporation, cell squeezing, lipofection, or chemicals (e.g., cell penetrating peptides).

“Coronavirus” as used herein refers to members of the family Coronaviridae, and therefore includes, but is not limited to, viruses such as Severe Acute Respiratory Syndrome Coronavirus (SARS-CoV), Middle East Respiratory Syndrome Coronavirus (MERS-CoV), and Severe Acute Respiratory Syndrome Coronavirus 2 (SARS-CoV-2; the causative agent of COVID-19).

### Agents Targeting 5′-Poly(U) Sequences

As described herein, the 5′-poly(U) tract of the negative-strand intermediates of coronaviruses, such as SARS-CoV-2, can be targeted and blocked using, for instance, a complementary nucleic acid sequence (as blocking the poly(U) tract prevents genomic and sub-genomic mRNA synthesis).

In some embodiments, the complementary nucleic acid sequence is a DNA, RNA, or DNA/RNA, and may or may not include one or more abasic sites, base analogs, and/or modified bases, for example. In certain aspects and embodiments, nucleic acid molecules may include one or more modifications (or chemical modifications). Such modifications can be in the nucleotide sugar, nucleotide base, nucleotide phosphate group and/or the phosphate backbone of a polynucleotide.

In certain embodiments, modifications as disclosed herein may be used to increase the in vivo stability of the nucleic acid molecule, particularly the stability in serum, and/or to increase bioavailability of the molecules. Non-limiting examples of modifications include, without limitation, internucleotide or internucleoside linkages; deoxynucleotides or dideoxyribonucleotides at any position and strand of the nucleic acid molecule; nucleic acid (e.g., ribonucleic acid) with a modification at the 2′-position preferably selected from an amino, fluoro, methoxy, alkoxy and alkyl; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other modified nucleotides may include, for example, 3′-deoxyadenosine, 3′-azido-3′-deoxythymidine, 2′,3′-dideoxyinosine, 2′,3′-dideoxy-3′-thiacytidine, 2′,3′-didehydro-2′,3′-dideoxythymidi-ne and the monophosphate nucleotides of 3′-azido-3′-deoxythymidine, 2′,3′-dideoxy thiacytidine and 2′,3′-didehydro-2′,3′-dide-oxythymidine.

Also contemplated herein are locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides. Chemical modifications also include unlocked nucleic acids, or UNAs, which are non-nucleotide, acyclic analogues, in which the C2′-C3′ bond is not present.

Chemical modifications also include terminal modifications on the 5′ and/or 3′ part of the nucleic acids, and are also known as capping moieties. Such terminal modifications are selected from a nucleotide, a modified nucleotide, a lipid, a peptide, and a sugar. Chemical modifications also include “six membered ring nucleotide analogs.” Examples of six-membered ring nucleotide analogs include hexitol and altritol nucleotide monomers.

Chemical modifications also include “mirror” nucleotides which have a reversed chirality as compared to normal naturally occurring nucleotide. Mirror nucleotides may further include at least one sugar or base modification and/or a backbone modification. Mirror nucleotides include, for example, L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboudenosine-3′-phosphate (mirror rA); L-ribocytidine-3′-phosphate (mirror rC); L-riboguanosine-3′-phosphate (mirror rG); and L-ribouracil-3′-phosphate (mirror dU).

In some embodiments, modified ribonucleotides include modified deoxyribonucleotides, for example 5′OMe DNA (5-methyl-deoxyriboguanosine-3′-phosphate); PACE (deoxyriboadenosine 3′ phosphonoacetate, deoxyribocytidine 3′ phosphonoacetate, deoxyriboguanosine 3′ phosphonoacetate, deoxyribothymidine 3′ phosphonoacetate).

Nucleobases of the nucleic acids disclosed herein may include unmodified deoxyribonucleotides and ribonucleotides (purines and pyrimidines) such as adenine, guanine, cytosine, thymidine, and uracil. Nucleobases can be modified with natural and synthetic nucleobases, such as thymine, xanthine, hypoxanthine, inosine, 2-aminoadenine, 6-methyl and other alkyl derivatives of adenine and guanine, any “universal base” nucleotides; 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 8-halo, amino, thiol, thioalkyl, hydroxyl and other 8-substituted adenines and guanines, 5-trifluoromethyl and other 5-substituted uncials and cytosines, 7-methylguanine, deazapurines, heterocyclic substituted analogs of purines and pyrimidines, e.g., aminoethoxy phenoxazine, derivatives of purines and pyrimidines (e.g., 1-alkyl-, 1-alkenyl-, heteroaromatic- and 1-alkynyl derivatives) and tautomers thereof, 8-oxo-N6-methyladenine, 7-diazaxanthine, 5-methylcytosine, 5-methyluracil, 5-(1-propynyl)uracil, 5-(1-propynyl) cytosine and 4,4-ethanocytosine). Other examples of suitable bases include non-purinyl and non-pyrimidinyl bases such as 2-aminopyridine and triazines.

Sugar moieties in nucleic acids disclosed herein may include 2′-hydroxyl-pentofuranosyl sugar moiety without any modification. Alternatively, sugar moieties can be modified such as, 2′-deoxy-pentofuranosyl sugar moiety, D-ribose, hexose, modification at the 2′ position of the pentofuranosyl sugar moiety such as 2′-O-alkyl (including 2′-O-methyl and 2′-O-ethyl). i.e., 2′-alkoxy, 2′-amino, 2′-O-allyl, 2′-S-alkyl, 2′-halogen (including 2′-fluoro, chloro, and bromo), 2′-methoxyethoxy, 2′-O-methoxyethyl, 2′-O-2-methoxyethyl, 2′-propargyl, 2′-propyl, ethynyl, ethenyl, propenyl, CF, cyano, imidazole, carboxylate, thioate.

In some embodiments, the pentafuranosyl ring may be replaced with acyclic derivatives lacking the C2′-C3′-bond of the pentafuranosyl ring. For example, acyclonucleotides may substitute a 2-hydroxyethoxymethyl group for-the 2′-deoxyribofuranosyl sugar normally present in dNMPs.

The nucleoside subunits of the nucleic acid disclosed herein may be linked to each other by phosphodiester bond. The phosphodiester bond may be optionally substituted with other linkages. For example, phosphorothioate, thiophosphate-D-ribose entities, triester, thioate, 2′-5′ bridged backbone, PACE, 3′-(or -5′)deoxy-3′-(or -5′)thio-phosphorothioate, phosphorodithioate, phosphoroselenates, 3′-(or -5′)deoxy phosphinates, borano phosphates, 3′-(or -5′)deoxy-3′-(or 5′-)amino phosphoramidates, hydrogen phosphonates, phosphonates, borano phosphate esters, phosphoramidates, alkyl or aryl phosphonates and phosphotriester modifications such as alkylphosphotriesters, phosphotriester phosphorus linkages, 5′-ethoxyphosphodiester, P-alkyloxyphosphotnester, methylphosphonate, and nonphosphorus containing linkages for example, carbonate, carbamate, silyl, sulfur, sulfonate, sulfonamide, formacetal, thioformacetyl, oxime, methyleneimino, methylenemethylimino, methylenehydrazo, methylenedimethylhydrazo and methyleneoxymethylimino linkages.

Nucleic acid molecules disclosed herein may include a peptide nucleic acid (PNA) backbone. The PNA backbone is includes repeating N-(2-aminoethyl)-glycine units linked by peptide bonds. The various bases such as purine, pyrimidine, natural and synthetic bases are linked to the backbone by methylene carbonyl bonds.

Modifications can be made at terminal phosphate groups. Non-limiting examples of different stabilization chemistries can be used, e.g., to stabilize the 3′-end of nucleic acid sequences, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6)3′-glyceryl; (7) [3′-5′]-3′-deoxyribonucleotide; (8) [3′-3′]-deoxyribonucleotide; (9) [5′-2′]-deoxyribonucleotide; and (10) [5′-3′]-dideoxyribonucleotides. In addition to unmodified backbone chemistries can be combined with one or more different backbone modifications described herein.

Modified nucleotides and nucleic acid molecules as provided herein may include conjugates, for example, a conjugate covalently attached to the nucleic acid molecule. The conjugate may be covalently attached to a nucleic acid molecule via a linker. In one embodiment, a conjugate molecule may include a molecule that facilitates delivery of a nucleic acid molecule into a cell, such as, for example, into a particular type of cell, or into a particular intracellular compartment or vesicle within a cell.

In some embodiments, the nucleic acid contains DNA, and contains a poly(A) or oligo(A) region that hybridizes to the 5′-poly(U) sequences.

The poly(A) or oligo(A) region may be about 100 or less, about 90 or less, about 80 or less, about 70 or less, about 60 or less, about 50 or less, about 40 or less, about 30 or less, about 25 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, about 3 or less, or about 2 or less, bases in length.

To further enhance targeting and specificity, and to further reduce off-target effects, the 5′-poly(U) sequences may be targeted using a polynucleotide that comprises a poly(A) sequence and which further comprises a 5′-end linker region that comprises a sequence complementary to a sequence at the 5′-end of the negative-strand RNA that starts immediately after the poly(U) stretch. In this configuration, both the poly(A) sequence and the 5′-end linker region sequence will hybridize to coronavirus negative-strand intermediates, such as SARS-CoV-2 negative-strand intermediates.

In other embodiments, the 5′-poly(U) stretch may be blocked using an antibody against poly(U). The antibody may be a fragment and/or a derivative of an antibody. Examples of antibody fragments include F(ab′)2, Fab, and Fv. Examples of antibody derivatives include: antibodies to which an amino acid mutation has been introduced in its constant region; antibodies in which the domain arrangement of the constant regions has been modified; antibodies having two or more Fc's per molecule; antibodies consisting only of a heavy chain or only of a light chain; antibodies with modified glycosylation; bispecific antibodies; conjugates of antibodies or antibody fragments with compounds or proteins other than antibodies; antibody enzymes; nanobodies; tandem scFv's; bispecific tandem scFv's; diabodies; and VHHs. The term “antibody” as used herein encompasses such fragments and/or derivatives of antibodies, unless otherwise specified.

The term “monoclonal antibody” conventionally means antibody molecules obtained from a clone derived from a single antibody-producing cell, i.e., a single variety of antibody molecules having a combination of VH and VL with specific amino acid sequences. A monoclonal antibody can also be produced via genetic engineering procedure, by preparing a nucleic acid molecule having a gene sequence encoding the amino acid sequence of the monoclonal antibody protein. A person skilled in the art would also be familiar with techniques for modifying a monoclonal antibody using genetic information about, e.g., H chains, L chains, variable regions thereof, and CDR sequences thereof to thereby improve the binding ability and specificity of the antibody, and techniques for preparing an antibody suitable for a therapeutic agent by altering an animal antibody such as a mouse antibody into a human-type antibody. A human-type monoclonal antibody can also be prepared by sensitizing a non-human transgenic animal carrying a human antibody gene to an antigen, such as poly(U).

In other embodiments, low molecular weight compounds or proteins that target the 5′-poly(U) stretch in the negative-strand intermediates may be used. Such low molecular weight compounds or proteins may be identified using, for example, library screening, or in silico modeling.

In other embodiments, aptamers that target the 5′-poly(U) stretch in the negative-strand intermediates may be used. Such aptamers may be identified using, for example, library screening, or a Systematic Evolution of Ligands by EXponential enrichment (SELEX) method.

### Delivery of Therapeutic Products and Compositions

Delivery of agents that target the 5′-poly(U) sequence may be achieved by a number of methods known to one of ordinary skill in the art. In some embodiments, these agents can be directly introduced into cells. Non-limiting methods to introduce these components into a cell include microinjection, electroporation, nucleofection, lipofection, particle gun technology, and microprojectile bombardment.

Agents that target the 5′-poly(U) region, such as nucleic acid molecules, may be delivered to cells using a delivery vehicle, such as a lipid vesicle or carrier. Agents, including nucleic acid molecules, may be delivered or administered to a subject by direct application of the agent (such as a nucleic acid molecule) with a carrier or diluent or any other delivery vehicle that acts to assist, promote or facilitate entry into a cell, including viral sequences, viral particular, liposome formulations, lipofectin or precipitating agents and the like.

Agents that target the 5′-poly(U) sequence, such as nucleic acid molecules, can also be administered to cells by a variety of methods known to those of skill in the art, including, but not limited to, encapsulation in liposomes, by iontophoresis, or by incorporation into other vehicles, such as biodegradable polymers, hydrogels, cyclodextrins, poly(lactic-co-glycolic)acid (PLGA) and PLCA microspheres, biodegradable nanocapsules, and bioadhesive microspheres, or by proteinaceous vectors.

In some embodiments, agents that target the 5′-poly(U) sequence, such as nucleic acid molecules, can be administered together with polyethylenimine (PEI) to facilitate delivery. In some embodiments, the PEI is a branched PEI. In other embodiments, agents that target the 5′-poly(U) sequence, such as nucleic acid molecules, can be administered together with a linear poly methacrylate cationic polymer, such as block copolymer of poly(oligoethylene oxide methacrylate), or block copolymer of poly(dimethylaminoethyl methacrylate).

Agents that target the 5′-poly(U) sequence, such as nucleic acid molecules, can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly(ethylene glycol) lipids (PEG-modified, or long-circulating liposomes or stealth liposomes). These formulations offer a method for increasing the accumulation of drugs in target tissues.

Agents that target the 5′-poly(U) sequence, such as nucleic acid molecules, can be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI), for example.

Agents that target the 5′-poly(U) sequence, such as nucleic acid molecules, can be complexed with membrane disruptive agents. The membrane disruptive agent or agents and the nucleic acid molecule may also be complexed with a cationic lipid or helper lipid molecule.

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

Compositions, methods and kits disclosed herein may include an expression vector that includes a nucleic acid sequence encoding at least one nucleic acid molecule of the invention in a manner that allows expression of the nucleic acid molecule. Methods of introducing nucleic acid molecules or one or more vectors capable of expressing the nucleic acid molecule in the environment of the cell will depend on the type of cell and the make up of its environment.

Nucleic acid molecules that hybridize to 5′-poly(U)-containing RNAs of coronavirus (e.g., from SARS-CoV-2) may be expressed from transcription units inserted into DNA or RNA vectors. Recombinant vectors can be DNA plasmids or viral vectors. Nucleic acid molecule-expressing viral vectors can be constructed based on, but not limited to, adeno-associated virus, retrovirus, adenovirus, or alphavirus. The recombinant vectors capable of expressing the nucleic acid molecules can be delivered as described herein, and persist in target cells. Alternatively, viral vectors can be used that provide for transient expression of nucleic acid molecules. Such vectors can be repeatedly administered as necessary. Delivery of nucleic acid molecule-expressing vectors can be systemic, such as by intravenous or intramuscular administration.

An expression vector may include one or more of the following: a) a transcription initiation region (e.g., eukaryotic pol I, II or III initiation region); b) a transcription termination region (e.g., eukaryotic pol I, II or III termination region); c) an intron and d) a nucleic acid sequence encoding at least one of the nucleic acid molecules, wherein said sequence is operably linked to the initiation region and the termination region in a manner that allows expression and/or delivery of the nucleic acid molecule.

Transcription of the nucleic acid molecule sequences can be driven from a promoter for eukaryotic RNA polymerase I (pol I). RNA polymerase II (pol II), or RNA polymerase III (pol III).

Polymeric nanocapsules or microcapsules facilitate transport and release of the encapsulated or bound agents, such a nucleic acids, into a cell. They include polymeric and monomeric materials, such as polybutylcyanoacrylate. Nucleic acid molecules may also be formulated as a microemulsion. A microemulsion is a system of water, oil and amphiphile which is a single optically isotropic and thermodynamically stable liquid solution. Delivery formulations can also include water soluble degradable crosslinked polymers.

The useful dosage to be administered and the particular mode of administration will vary depending upon such factors as the cell type, or for in vivo use, the age, weight and the particular animal and region thereof to be treated, the particular agent (e.g., a nucleic acid) and delivery method used, the therapeutic or diagnostic use contemplated, and the form of the formulation, for example, suspension, emulsion, micelle or liposome, as will be readily apparent to those skilled in the art.

When lipids are used to deliver the nucleic acid, the amount of lipid compound that is administered can vary and generally depends upon the amount of nucleic acid being administered. For example, the weight ratio of lipid compound to nucleic acid is preferably from about 1:1 to about 30:1, with a weight ratio of about 5:1 to about 10:1 being more preferred.

A suitable dosage unit of nucleic acid molecules may be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day.

The administered agents that target the 5′-poly(U)-containing RNAs (e.g., from SARS-CoV-2) can be used to treat individuals infected with, or suspected of being infected with, a coronavirus, such as SARS-CoV-2.

The administration of the agents and compositions of the present disclosure to subjects may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. The agents or compositions can be administrated in one or more doses. In some embodiments, an effective amount is administrated as a single dose. In other embodiments, an effective amount is administrated as more than one dose, over a period time. The determination of optimal ranges of effective amounts of a given cell type for a particular disease or condition is within the skill of those in the art.

Treatments of the present disclosure can be provided in conjunction with, before, or after, vaccination, and/or supportive care, such as supplemental oxygen, mechanical ventilation, and steroids.

### Detection Methods

By targeting a target unique to coronaviruses, such as SARS-CoV-2, the 5′-poly(U) stretch in the negative-strand intermediates can be used as a marker for a coronavirus infection, such as SARS-CoV-2 infection; and/or as a marker for the presence of a coronavirus, such as SARS-CoV-2, in a sample. Therefore, nucleic acids and compositions of the present disclosure also have utility in the field of coronavirus detection, diagnosis, and management, such as SARS-CoV-2 detection, diagnosis, and management.

Agents as described above for use in treatment can also be used in such detection methods, as long as their binding to the 5′-poly(U) stretch leads to a detectable change. For instance, poly(A)-containing nucleic acid molecules that specifically hybridize to the 5′-poly(U) stretch can be labeled, and their hybridization to the 5′-poly(U) stretch can be determined by a change in a detectable signal. For instance, poly(A)-containing nucleic acid molecules can be labeled with a fluorescent dye or label.

Fluorescence broadly refers to the process or the result of the emission of light by a substance that has absorbed light or other electromagnetic radiation. Fluorophores or fluorescent dyes are chemical compounds or moieties that can re-emit light upon light excitation. Fluorophores typically contain several combined aromatic groups, or plane or cyclic molecules with several πbonds. A fluorophore absorbs light energy of a specific wavelength and re-emits light at a longer wavelength. When a fluorophore is excited at a particular wavelength, it is promoted to an excited state. In the absence of a quencher, the excited dye emits light in returning to the ground state. When a quencher is present in physical proximity, the excited fluorophore can return to the ground state by transferring its energy to the quencher, without the emission of light.

Different types of quenchers exist. One quenching mechanism relies on the ability of the fluorophore to transfer energy to a second fluorophore by fluorescence resonance energy transfer (FRET). This returns the fluorophore to the ground state and generates the quencher excited state. The quencher then returns to the ground state through emissive decay (fluorescence). In order for this to happen, the emission spectrum of the fluorophore must overlap with the absorption spectrum of the second fluorophore (quencher). One example of such the fluorophore/quencher pair is fluorescein (used as the fluorescent reporter dye) and rhodamine as the quencher (FAM/TAM probes).

However, quencher fluorescence can increase background noise due to overlap between the quencher and reporter fluorescence spectra. Dark quenchers are dyes with no native fluorescence. Dark quenchers return from the excited state to the ground state via non-radiative decay pathways, without the emission of light. In dark decay, energy is given off via molecular vibrations (heat). With the typical μM or less concentration of probe, the heat from radiationless decay is too small to affect the temperature of the solution. Dark quenchers do not occupy an emission bandwidth and allow multiplexing, when two or more reporter-quencher probes are used together. BHQ quenchers are examples of dark quenchers. Thus, a dark quencher, a substance or moiety that absorbs excitation energy from a fluorophore and dissipates the energy as heat, may be used in embodiments of the present disclosure.

Non-limiting examples of detection methods include optical detection, spectroscopic detection, electrostatic detection and electrochemical detection. Accordingly, an agent or nucleic acid molecule described herein may be detected by detecting signals (e.g., signals indicative of an optical property, a spectroscopic property, an electrostatic property or an electrochemical property of the agent, nucleic acid molecule, or an associated detectable label) that are indicative of the presence or absence of the nucleic acid molecule. Optical detection methods include, but are not limited to, visual inspection (e.g., detection via the eye, observing an optical property or optical event without the aid of an optical detector), fluorimetry, chemiluminescence imaging, fluorescence resonance energy transfer (FRET) and UV-vis light absorbance. Spectroscopic detection methods include, but are not limited to, mass spectrometry, nuclear magnetic resonance (NMR) spectroscopy, Raman spectroscopy, and infrared spectroscopy. Electrostatic detection methods include, but are not limited to, gel based techniques, such as, for example, gel electrophoresis (e.g., agarose gel or polyacrylamide gel electrophoresis).

In some embodiments, detection of a nucleic acid molecule described herein may be achieved with the aid of a detectable label, such as a dye, protein, or conjugate. A detectable label may be linked or coupled with a nucleic acid molecule covalently and/or non-covalently (e.g., including intercalation of a double-stranded nucleic acid molecule). Non-limiting examples of detectable labels include optically-responsive species (e.g., optically-responsive dyes, optically-responsive oligonucleotide probes (e.g., TaqMan probes, TaqMan Tamara probes, TaqMan MGB probes, Lion probes, molecular beacons)) and radiolabels.

In some embodiments, one or more of the reagents (e.g., a nucleic acid probe) described herein can comprise a detectable label and/or comprise the ability to generate a detectable signal (e.g., by catalyzing a reaction converting a compound to a detectable product). Detectable labels can comprise, for example, a light-absorbing dye, a fluorescent dye, or a radioactive label. Detectable labels, methods of detecting them, and methods of incorporating them into reagents (e.g., antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels can be primary labels (where the label comprises a moiety that is directly detectable or that produces a directly detectable moiety); or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to, radioisotopes, bioluminescent compounds, chromophores, antibodies, chemiluminescent compounds, fluorescent compounds, metal chelates, and enzymes.

In some embodiments, a detectable label may be an optically-responsive dye (e.g., a fluorescent dye) that generates (or fails to generate a signal) when subjected to the appropriate conditions. Non-limiting examples of dyes include SYBR green, SYBR blue, DAPI, propidium iodine, Hoeste, SYBR gold, EvaGreen, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), SYBR Green, Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In certain embodiments, the 5′-poly(U) stretch in the negative-strand intermediates can be detected using a nucleic amplification reaction that amplifies a nucleic acid sequence that contains a sequence corresponding to all or a part of the poly(U) stretch (e.g., polymerase chain reaction, RT-PCR).

For instance, in some embodiments, a primer comprising a portion of the SARS-CoV-2 genome upstream of the poly(A) sequence (or a primer containing the equivalent cDNA sequence) can be used as a primer to bind to the negative-strand intermediates to initiate first strand synthesis. In some embodiments, this primer sequence lies within the 3′-untranslated region (3′-UTR) of the SARS-CoV-2 genome. In some embodiments, this primer sequence comprises the sequence of SEQ ID NO: 3. Second-strand synthesis can then be performed using, for example, an oligo(dT) primer.

Other known amplification methods which can be utilized herein include, but are not limited to, “NASBA” or “35R” techniques; Q-beta amplification; strand displacement amplification; target mediated amplification; ligase chain reaction (LCR); self-sustained sequence replication (SSR); and transcription amplification.

Methods for detecting, characterizing, and/or quantitating, nucleic acid sequences are known to persons skilled in the art, and include, but are not limited to, for example, PCR procedures, RT-PCR, quantitative PCR or RT-PCR, Northern blot analysis, differential gene expression, RNA protection assay, microarray analysis, hybridization methods, serial analysis of gene expression (SAGE), hybridization based on digital barcode quantification assays, multiplex RT-PCR, digital drop PCR (ddPCR), qRT-PCR, qPCR, UV spectroscopy, DNA sequencing, RNA sequencing, next-generation sequencing, including RNAseq, lysate-based hybridization assays utilizing branched DNA signal amplification, such as the QuantiGene 2.0 Single Plex, and branched DNA analysis methods.

Non-limiting examples of nucleic acid sequencing techniques, e.g., for DNA sequencing and RNA sequencing, include Maxam-Gilbert sequencing, Sanger sequencing (i.e., chain-termination), sequencing-by-synthesis (SBS), sequencing-by-ligation, pyrosequencing, single-molecule real-time sequencing, MiSeq sequencing, massively parallel signature sequencing (MPSS), polony sequencing, 454 sequencing, nanopore sequencing. The present disclosure also encompasses, but is not limited to, next-generation sequencing technologies.

Non-limiting examples of next-generation sequencing technologies include, for example, Ion Torrent, Illumina, SOLiD, 454; Massively Parallel Signature Sequencing solid-phase, reversible dye-terminator sequencing; and DNA nanoball sequencing. Digital barcode quantification assays can include the BeadArray (Illumina), the xMAP systems (Luminex), the nCounter (Nanostring), the High Throughput Genomics (HTG) molecular, BioMark (Fluidigm), or the Wafergen microarray. Assays can include DASL (Illumina), RNA-Seq (Illumina), TruSeq (Illumina), SureSelect (Agilent), Bioanalyzer (Agilent) and TaqMan (ThermoFisher).

In general, PCR describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e., each primer is specifically designed to be complementary to a strand of the genomic locus to be amplified. The mRNA level of a gene can be determined by reverse-transcription (RT) PCR and by quantitative RT-PCR (QRT-PCR), or real-time PCR methods. Methods of RT-PCR and QRT-PCR are well known in the art. The nucleic acid sequences of exemplary marker genes are set forth herein. Accordingly, a skilled artisan can design an appropriate primer based on the disclosed sequences for determining the mRNA level of the respective marker gene.

Nucleic acid and ribonucleic acid (RNA) molecules can be isolated from a sample using any of a number of procedures, which are well-known in the art, the particular isolation procedure chosen being appropriate for the particular biological sample.

### Experimental

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for.

Unless indicated otherwise, temperature is in degrees Centigrade and pressure is at or near atmospheric. It should be understood that these Examples are given by way of illustration only and are not intended to limit the scope of what the inventor regards as various embodiments of the present invention. Not all of the following steps set forth in each Example are required nor must the order of the steps in each Example be as presented.

### Example 1

### Design and Synthesis of Nucleic Acid Molecules Targeting the 5′- Poly(U) Tract of SARS-CoV-2

This Example illustrates the design and synthesis of nucleic acid molecules targeting the 5′-poly(U) tract of SARS-CoV-2. Poly-deoxyadenylate (poly(dA))-containing nucleic acids were designed and synthesized; these nucleic acids contained, in addition to the poly(dA) sequence, a 5′—end linker region complementary to a sequence at the 5′-end of the SARS-CoV-2 negative-strand RNA intermediates that starts immediately after the poly(U) stretch. This additional linker region was included to provide even further specificity to the binding to the 5′-end of the poly(U) of the SARS-CoV-2 virus.

Two different nucleic acid molecules were synthesized. The first, termed “A25” (5′-GGAGAATGACA(25) -3′; SEQ ID NO: 1) contained a 10-nucleotide linker sequence (GGAGAATGAC; nucleotides 1-10 of SEQ ID NO: 1) attached to the 5′-end of a 25-mer poly(dA) sequence. The second, termed “A25L” (5′- A(2s)GGAGAATGACA(25) -3′; SEQ ID NO: 2) contained a 10-nucleotide linker sequence (GGAGAATGAC; nucleotides 1-10 of SEQ ID NO: 1) sandwiched between two copies of a 25-mer poly(dA) sequence.

The linker sequence was selected to distinguish the poly(U) at the 5′ end of the poly(U) of the SARS-CoV-2 virus from any possible 3′-poly(U) sequences (which can be present in some small host RNAs). The linker sequence was complementary to a sequence at the 5′-end of the negative-strand RNA of SARS-CoV-2 that starts immediately after the poly(U) stretch.

### Example 2

### Analysis of A25 and A25L on SARS-CoV-2 Replication

The effect of the A25 and A25L nucleic acid molecules on SARS-CoV-2 replication was determined using a virus-induced cytopathic effect (CPE) assay (Primary CPE assay). In this assay, confluent (or near-confluent) cell culture monolayers of Vero 76 cells were prepared in 96-well disposable microplates the day before testing. The cells were maintained in Minimum Essential Medium (MEM) supplemented with 5% fetal bovine serum (FBS).

For antiviral assays, the same medium was used, but with FBS reduced to 2% and supplemented with 50-μg/ml gentamicin. The nucleic acids were dissolved in DMSO or saline, and were prepared at four serial login concentrations (0.1, 1.0, 10, and 100 μg/ml). Five microwells were used per dilution: three for infected cultures, and two for uninfected toxicity cultures. Controls for the experiment consisted of, for each plate, six microwells that were infected and not treated (virus controls); and six microwells that were untreated and uninfected (cell controls). A known active drug was tested in parallel as a positive control drug, using the same method applied to the test nucleic acids. The positive control was tested with every test run.

Growth media was removed from the cells, and the test nucleic acids (A25 and A25L) were applied in 0.1 ml volumes to wells at 2× concentration. Virus, at ˜60 CCID₅₀ (50% cell culture infectious dose) in a 0.1 ml volume, was added to the wells designated for virus infection. Medium devoid of virus was placed in toxicity control wells and cell control wells. The plates were then incubated at 37° C., with 5% CO₂, until marked CPE (>80% CPE) was observed in the virus control wells. The plates were then stained with 0.011% neutral red, for approximately two hours at 37° C. in a 5% CO₂ incubator. The neutral red medium was then removed by complete aspiration, and the cells were rinsed once with phosphate buffered saline (PBS) to remove residual dye. The PBS was then completely removed, and the incorporated neutral red was eluted with 50% Sorensen's citrate buffer/50% ethanol for at least 30 minutes. Neutral red dye penetrates into living cells, and thus, the more intense the red color, the larger the number of viable cells present in the wells. The dye content in each well was quantified using a spectrophotometer (at 540 nm wavelength). The dye content in each set of wells was converted to a percentage of dye present in untreated control wells, using a Microsoft Excel computer-based spreadsheet, and normalized based on the virus control. The 50% effective (EC₅₀, the compound concentration that reduces viral replication by 50%) concentrations were then calculated by regression analysis.

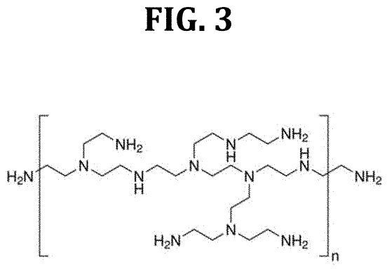

The A25 and A25L nucleic acids were tested with and without different polymers, to determine whether the effect was enhanced when the A25 and A25L nucleic acids were administered as a complex with the polymer. Three different polymers were tested: (1) branched polyethylenimine (“bPEI,” molecular weight ˜10 kDa); (2) jetOPTIMUS PEI (www.polyplus-transfection.com/products/jetoptimus; “Polymer 1,” molecular weight ˜10 kDa); and (3) linear poly methacrylate cationic polymer (“Polymer 2,” molecular weight ˜10 kDa). The structure of bPEI is shown in FIG. 3.

The following Table depicts the effect of the A25 or A25L oligos on the CPE induced by SARS-CoV-2.

| | Visual | | Neutral Red | |
|---|---|---|---|---|
| | (Cytopathic effect/ | | (Cytopathic effect/ | |
| Treatment | Toxicity) EC₅₀ | | Toxicity) EC₅₀ | |
| A25L bPEI | 6.3 | mg/ml | 8.4 | mg/ml |
| A25 bPEI | 6.3 | mg/ml | 5.1 | mg/ml |
| A25L (no complex with bPEI) | >20 | mg/ml | >20 | mg/ml |
| A25 (no complex with bPEI) | >20 | mg/ml | >20 | mg/ml |
| bPEI | >20 | mg/ml | >20 | mg/ml |
| A25-Polymer2 | 0.68 | mM | 1.0 | mM |
| A25L-Polymer1 | 0.68 | mM | 0.76 | mM |
| Polymer2 | >32 | mg/ml | >34 | mg/ml |

The results shown in the above Table demonstrate that the poly(dA) oligos (A25 and A25L) markedly inhibited SARS-CoV-2 proliferation in Vero-76 cells.

The bPEI alone, or A25 or A25L (with bPEI or alone) were used at 0.02 to 20 mg/ml concentrations to calculate the EC₅₀. Since the A25 and A25L nucleic acids were complexed with bPEI in some samples, bPEI without either A25 or A25L was used as a control to exclude an inhibitory effect caused solely by bPEI.

The EC₅₀ values depicted in Table 1 show that A25 or A25L, complexed with bPEI, were almost 4-fold more effective in inhibiting SARS-CoV-2 inhibition compared to that of the transfection vehicle alone. A similar level of inhibition (to A25-bPEI and A25L-bPEI) was observed when A25 or A25L was complexed with either Polymer 1 or Polymer 2 (considering the molecular weights of A25 and A25L (˜12200 g/mol and ˜20450 g/mol, respectively), the 0.68 mM EC₅₀ value for A25-Polymer 2 and A25L-Polymer 1 was converted to mg/ml to compare the effect with the polymer alone); the conversion showed that the A25-Polymer 2 and A25L-Polymer 1 treatments inhibited CPE induced by SARS-CoV-2 by almost 4-fold as compared to polymer alone.

### Example 3

### Detection of SARS-CoV-2 Infection by Detecting the 5′-Poly(U) Stretch of SARS-CoV-2 Negative-Strand RNAs

Since the negative-strand RNAs of coronaviruses are synthesized immediately after infection, detection of the 5′-poly(U) stretch of SARS-CoV-2 negative-strand RNAs represents a rapid-arising marker for SARS-CoV-2 infection. By way of example, specific cDNA amplification of the SARS-CoV-2 negative-strand RNAs would be possible using a cDNA synthesis primer having the sequence 5′ CATTAGGGAGGACTTGAAAGAGC 3′ (SEQ ID NO: 3; corresponding to nucleotides 29696-29718 of the SARS-CoV-2 positive strand genome RNA), together with an oligo(dT) primer. These two primers, when used in a nucleic acid amplification reaction, would selectively amplify the region corresponding to nucleotides 29696-29903 of the SARS-CoV-2 positive strand genome RNA

(CATTAGGGAGGACTTGAAAGAGCCACCACATTTTCAC
CGAGGCCACGCGGAGTACGATCGAGTGTACAGTGAACA
ATGCTAGGGAGAGCTGCCTATATGGAAGAGCCCTAATG
TGTAAAATTAATTTTAGTAGTGCTATCCCCATGTGATT
TTAATAGCTTCTTAGGAGAATGACAAAAAAAAAAAAAA
AAAAAAAAAAAAAAAAAAA; SEQ ID NO: 4).

1. An agent that binds to a negative-strand ribonucleic acid (RNA) molecule produced by a coronavirus, preferably SARS-CoV-2, wherein said agent is capable of binding to a 5′-poly(U) stretch in said negative-strand RNA molecule, and wherein said agent is selected from the group consisting of a nucleic acid, an antibody, an aptamer, a protein, and a low molecular weight compound.

2. A nucleic acid molecule that binds to a negative-strand ribonucleic acid (RNA) molecule produced by a coronavirus, preferably SARS-CoV-2, wherein said nucleic acid molecule comprises a poly(A) stretch that is between 5-50 bases in length, and wherein said poly(A) stretch is capable of hybridizing to a 5′-poly(U) stretch in said negative-strand RNA molecule.

3. The nucleic acid molecule of claim 2, wherein the nucleic acid molecule further comprises a 5′-end linker region that comprises a sequence complementary to a sequence at the 5′-end of the negative-strand RNA that starts immediately after the poly(U) stretch.

4. The nucleic acid molecule of claim 3, wherein the linker sequence comprises GGAGAATGAC (nucleotides 1-10 of SEQ ID NO: 1).

5. The nucleic acid molecule of claim 4, wherein said nucleic acid molecule comprises a nucleotide sequence selected from the group consisting of SEQ ID NO: 1 and SEQ ID NO: 2.

6. A composition comprising the nucleic acid molecule of claim 2, wherein said composition further comprises a delivery vehicle.

7. The composition of claim 6, wherein the delivery vehicle is a polymer.

8. The composition of claim 7, wherein the polymer is selected from the group consisting of a polyethylenimine polymer and a linear poly methacrylate cationic polymer.

9. The composition of claim 8, wherein the polyethylenimine polymer is a branched polyethylenimine polymer.

10. The composition of claim 6, wherein the delivery vehicle is selected from the group consisting of a lipid, a liposome, a hydrogels, a cyclodextrin, poly(lactic-co-glycolic)acid (PLGA), a microsphere, a nanocapsule, and a protein.

11. The nucleic acid molecule of claim 2, wherein the nucleic acid molecule contains at least one of a modified base, a base analog, and an abasic site.

12. The nucleic acid molecule of any of claim 2, wherein the nucleic acid molecule is conjugated to a heterologous molecule.

13. The nucleic acid molecule of claim 12, wherein the heterologous molecule is a detectable label.

14. The nucleic acid molecule of claim 12, wherein the heterologous molecule is a molecule that targets the nucleic acid molecule to a cell or intracellular compartment thereof.

15. A method for treating a coronavirus infection, preferably SARS-CoV-2 infection, comprising administering to a subject in need thereof the nucleic acid molecule of claim 2.

16. A method for treating a coronavirus infection, preferably SARS-CoV-2 infection, comprising administering to a subject in need thereof the composition of claim 6.

17. A method for treating a coronavirus infection, preferably SARS-CoV-2 infection, comprising administering to a subject in need thereof, an agent that binds to a 5′-poly(U) sequence in a negative-strand ribonucleic acid (RNA) molecule produced by a coronavirus, preferably SARS-CoV-2.

18. The method of claim 17, wherein said agent is selected from the group consisting of a nucleic acid, an antibody, an aptamer, a protein, and a low molecular weight compound.

19. A method of blocking a 5′-poly(U) sequence in a negative-strand ribonucleic acid (RNA) molecule produced by a coronavirus, preferably SARS-CoV-2, wherein said method comprises contacting the agent of claim 1 with a negative-strand ribonucleic acid (RNA) molecule produced by a coronavirus, preferably SARS-CoV-2.

20. A method of blocking a 5′-poly(U) sequence in a negative-strand ribonucleic acid (RNA) molecule produced by a coronavirus, preferably SARS-CoV-2, wherein said method comprises contacting the nucleic acid molecule of claim 2 with a negative-strand ribonucleic acid (RNA) molecule produced by a coronavirus, preferably SARS-CoV-2.

21. A method of blocking a 5′-poly(U) sequence in a negative-strand ribonucleic acid (RNA) molecule produced by a coronavirus, preferably SARS-CoV-2, wherein said method comprises contacting the composition of claim 6 with a negative-strand ribonucleic acid (RNA) molecule produced by a coronavirus, preferably SARS-CoV-2.

22. The nucleic acid of claim 2, wherein the poly(A) stretch is between 20-30 bases in length.

23. A method of detecting a coronavirus, preferably SARS-CoV-2, comprising detecting the presence of a negative-strand ribonucleic acid (RNA) molecule produced by a coronavirus, preferably SARS-CoV-2 in a sample, wherein said negative-strand RNA comprises a 5′-poly(U) stretch.

24. The method of claim 23, wherein said negative-strand RNA is detected using a nucleic acid amplification reaction, and wherein said nucleic acid amplification reaction amplifies a region that contains all or a part of said poly(U) stretch.

25. The method of claim 24, wherein said nucleic acid amplification reaction comprises use of an oligo(dT) primer.

26. The method of claim 25, wherein said nucleic acid amplification reaction further comprises use of an oligonucleotide primer comprising the sequence of SEQ ID NO: 3.

27. The method of claim 24, wherein said nucleic acid amplification reaction amplifies all or a portion of the region corresponding to SEQ ID NO: 4.

28. The nucleic acid of claim 2, wherein the nucleic acid comprises DNA, RNA, or DNA and RNA.

29. The nucleic acid of claim 28, wherein the nucleic acid comprises at least one of a modified base, a base analog, or an abasic site.


SEQUENCE LISTING (4 sequences)

SEQ ID NO: 1 - DNA, 35 bp, Artificial Sequence
  Synthetic polynucleotide sequence
ggagaatgacaaaaaaaaaaaaaaaaaaaaaaaaa

SEQ ID NO: 2 - DNA, 60 bp, Artificial Sequence
  Synthetic polynucleotide sequence
aaaaaaaaaaaaaaaaaaaaaaaaaggagaatgacaaaaaaaaaaaaaaaaaaaaaaaaa

SEQ ID NO: 3 - DNA, 23 bp, SARS-CoV-2
cattagggaggacttgaaagagc

SEQ ID NO: 4 - DNA, 208 bp, SARS-CoV-2
cattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa